(19) United States
(12) Patent Application Publication
(10) Pub. No.: US 2021/0338715 A1
(43) Pub. Date: Nov. 4, 2021

(54) PHARMACEUTICAL COMPOSITION FOR THE TREATMENT OF COVID-19

(71) Applicant: RADINOR, LLC, San Antonio, TX (US)

(72) Inventor: Andreas Ludwig Kalcker, Sennwald (CH)

(21) Appl. No.: 16/927,613

(22) Filed: Jul. 13, 2020

(30) Foreign Application Priority Data
Apr. 29, 2020 (CH) 0506/20

(51) Int. Cl. A61K 33/20; A61K 9/08; A61K 47/26; A61K 47/20; A61K 9/00; A01N 59/00; A61L 2/18; A61L 2/00; A61L 9/14

(52) U.S. Cl. CPC A61K 33/20; A61K 9/08; A61K 47/26; A61K 47/20; A61K 9/0053; A61L 2101/06; A01N 59/00; A61L 2/18; A61L 2/0088; A61L 9/14; A61K 9/0019

(57) ABSTRACT

The invention relates to a pharmaceutical composition based on a chlorine dioxide solution, for use as a therapeutic agent in the topical, oral, and/or parenteral treatment of viral infections of the human or animal body, caused by SARS-CoV-2 coronaviruses.

### TECHNICAL FIELD

The present invention relates to a pharmaceutical composition based on an aqueous chlorine dioxide solution, for the therapeutic treatment of infectious diseases caused by coronavirus, in particular COVID-19 caused by SARS-CoV2.

### STATE OF THE ART

A wide range of chemicals are used for surface disinfection, mostly oxidizing substances such as chlorine gas, ozone, hydrogen peroxide, chlorite, chlorate, or perchlorate solutions. These chemicals react non-specifically with organic material through the oxidation of in particular amino groups, sulfhydryl groups, double bonds, and aromatic systems. Their antibacterial and antiviral activity is substantially based on this effect.

In addition, chlorine dioxide has also been used as an effective, antimicrobial agent for almost a hundred years, with the applications so far mainly limited to surface disinfection and water hygiene.

Medical-therapeutic applications are also known, albeit to a surprisingly small extent, e.g. for external skin and wound disinfection, for root canal treatments in dentistry, mouthwashes against bad breath or candidiasis, and similar external and topical applications in humans and animals.

U.S. Pat. No. 5,252,343 describes in 1993 the successful treatment of mastitis in cows with a chlorine dioxide solution (CDL) by rinsing the teats on the udder of the cow, whereby in contrast to conventional antibiotic therapy, the milk of the animals treated with CDL did not have to be discarded, but could be continued to be used for consumption. This is because treatment with CDL apparently did not result in any pollutants of any kind and no degradation products of ClO2 that were potentially hazardous to health were found in relevant concentrations.

There is plenty of literature on the possible mechanisms of action of ClO2 in relation to proteins, amino acids, nucleic acids, polysaccharides, lipids, lipid membranes, bacteria, spores, viruses, fungi, unicellular organisms, and higher parasites. It is all the more surprising that this simple oxidant ClO2 is still not widely used in the medical and veterinary therapy of inflammation and infectious diseases. This may have something to do with an incomplete understanding of the physiology of immune defense in humans and animals that is widespread in the professional world, which means that many diseases are considered to be caused by free oxygen or nitrogen oxide radicals and the therapy is therefore aimed at eliminating such free radicals—also referred to in the specialist literature as “reactive oxidative species” (ROS)—by administering antioxidants.

In his review “Oxidative Shielding or Oxidative Stress?” from 2012 (The Journal of Pharmacology and Experimental Therapeutics, 2012, Vol. 342, No. 3, pp 608-618), Robert Naviaux tries to investigate this fact and shows that reactive oxidative species like free oxygen radicals are not the cause but rather the result of diseases. Oxidative shielding is a protective mechanism and should therefore not be a target for anti-oxidative therapy.

On the Possible Mechanism of Action of ClO2:

Even if the mechanism of action may not always be clarified in detail, the relevant specialist literature shows that chlorine dioxide, in contrast to chlorite and chlorate ions, does not indiscriminately attack any organic material, but selectively primarily proteins and amino acids with secondary and tertiary amines and/or sulfhydryl groups, especially cysteine, methionine, and glutathione, as well as aromatic amino acids such as tyrosine and tryptophan. In general, the specialist literature speaks of “electron-rich” regions of organic molecules (see, for example, Lee et al.; Water Research 44, (2010), 555-566), which are preferably oxidized by chlorine dioxide in one or more successive stages. The reaction products on the part of ClO2 are ultimately chloride ions, in other words NaCl in physiological solutions, and water.

In the light of the relevant specialist literature, reservations in parts of the specialist world regarding the therapeutic use of ClO2 seem to be based rather on an insufficient evaluation of existing specialist knowledge, perhaps also on certain prejudices. Relevant studies on model organisms in vitro show, for example, that antiviral effects are probably caused by the fact that, depending on the type of virus, either spikes are changed so that attachment to the host cell can no longer take place, or that the virus capsid is changed significantly, so that the immunogenicity is lost, or that certain steps in protein biosynthesis are negatively influenced, so that, for example, the transcription of the virus RNA or DNA no longer functions properly or the assembly of the virus particles is disturbed.

In the case of the effect on living unicellular organisms, for example the antibacterial and antifungal effect, the experts seem to have recognized that this microbicidal effect is primarily caused by a disruption of the cell membranes or the lipid membranes of important organelles by the action of ClO2, whereby, for example, damage to membrane proteins, such as tunnel proteins, causes the membranes to become non-specific to certain ions, which means that the cells, organelles, or, for example, bacterial or fungal spores, can no longer maintain their membrane potential and thus perish. There are also interesting reports on the signaling of small molecules such as ClO2, especially in relation to apoptosis induction, either directly or indirectly through the modulation of ATP, ADP, or GMP concentrations. Apoptosis in connection with immune defense is by no means seen as negative, but rather as the limited, targeted elimination of individual, infected cells in order to hinder or prevent a rapid multiplication and spread of the pathogens, for the benefit of the whole organism.

Noszticzius et al. describe in their paper “Chlorine Dioxide Is a Size-Selective Antimicrobial Agent” (Noszticzius Z, Wittmann M, Káty-Kullai K, Beregvári Z, Kiss I, et al., 2013, PLoS ONE 8(11): e79157. doi:10.1371/journal.pone.0079157) in detail why the administration of CDL selectively kills small organisms such as bacteria and viruses and leaves body cells undisturbed. Among other things, they approach the topic from the reaction kinetic side and very plausibly demonstrate that on the one hand certain proteins and amino acids are primarily attacked (see above) and on the other hand that the flooding of small entities such as bacteria using ClO2 in a germicidal amount is simply quicker than with much larger cells such as blood cells or somatic cells. In addition, eukaryotic cells have repair mechanisms against oxidative damage, especially those of sulfhydryl groups that viruses and bacteria lack.

In the light of the relevant specialist literature, reservations in parts of the specialist world regarding the therapeutic use of ClO2 seem to be based rather on an insufficient evaluation of existing specialist knowledge, perhaps also on certain prejudices.

It is an object of the present invention to take these ideas from Naviaux, on the one hand, and from Noszticzius et al. on the other hand into account and to provide a water-based composition which contains chlorine dioxide as an oxidative agent and is suitable and can be used for the therapy of coronavirus infections and the associated pathological conditions in humans, the focus being on the therapy of infections caused by SARS CoV2 and the associated disease COVID-19 in humans.

This object is achieved by a chlorine dioxide solution for the treatment of COVID-19 patients, as described in the independent claim. Further advantageous features of the invention are set out in the dependent claims.

### DESCRIPTION OF THE INVENTION

Chlorine dioxide (ClO2) is a yellowish gas that is very soluble in water at temperatures below 11 degrees Celsius. Surprisingly, it has not yet found its way into conventional pharmacopoeias as an active ingredient, although it is ideally suited for disinfection and is mandatory for storing blood bags for transfusions in many places. Its disinfectant, antimicrobial effect is primarily based on the oxidation of organic molecules, similar to how our own body functions in the context of the immunological defense against pathogens through oxidative denaturation in the course of phagocytosis by macrophages.

The recent COVID-19 coronavirus pandemic urgently requires solutions with new approaches. One such approach is the therapeutic use of chlorine dioxide (ClO2) in aqueous solution in low doses, either topically for external use on the skin, in particular for disinfecting the hands, and/or systemically via parenteral or intravenous use for therapeutic intervention in the case of an infection that has already occurred of the human body.

It is known that chlorine dioxide solution can be successfully used in human blood supplies against viruses such as HIV and other pathogens. In the course of numerous therapeutic uses, which were carried out on behalf of and in some cases under the guidance of the inventor by medical staff on infected individuals, it could be shown that chlorine dioxide solution can also be successfully used for the treatment of other viral infections, including for the treatment of infections by coronaviruses.

Chlorine dioxide inactivates the virus in a very short time through a selective oxidation process. This is done first by oxidative denaturation of the capsid proteins and subsequently also by oxidation of the RNA or DNA of the virus.

The use of chlorine dioxide for therapeutic purposes has been investigated by the inventor for over thirteen years and has found its way into several patent applications. A suitable chlorine dioxide solution can be prepared from any pharmacy as a magistral formulation and has been listed in a similar form in the old German drug code as “sodium chlorosum” (DAC N-055) since 1990.

To combat viral infections, either actively immunizing vaccines are typically used as a preventive measure or passively acting immune sera based on specific antiviral antibodies for acute therapy. This is apparently also the goal in the fight against the current coronavirus of the type SARS CoV2. Both measures are expensive and are not yet available for the treatment of COVID-19 patients. In addition, vaccination using an actively immunizing vaccine requires a healthy body that is able to provide the energy for the production of the body's own antibodies against the pathogen. Physically weakened individuals and those with an insufficiently functioning immune system are therefore not predestined for active immunization, the risk of a preventive vaccination would be too great.

For such individuals and for patients already infected by the coronavirus, the great advantage of chlorine dioxide (ClO2) in aqueous solution can be particularly effective. The chlorine dioxide solution does not burden the immune system, but—on the contrary—supports the immune system in at least two ways. Namely, on the one hand through the direct chemical attack on the virus particles and on the other hand through the provision of oxygen for ATP production in the mitochondria. In addition, measurements of venous blood gases have shown that after intravenous administration of chlorine dioxide solution, a significantly increased oxygen partial pressure in the test persons' venous blood was detectable. Chlorine dioxide administration also seems to have a positive effect on the oxygen supply to the lungs, i.e. to improve it.

It is possible that chlorine dioxide (ClO2) is virucidal against all types of viruses. So far, there have been no reports of any resistance to this type of oxidation. Not least because of this, this substance has been used in wastewater disinfection for a hundred years without generating any kind of resistance.

The fact that chlorine dioxide is also effective against coronavirus was already confirmed in 2005 by Barry Wintner et al. [Chlorine Dioxide, Part 1 A Versatile, High-Value Sterilant for the Biopharmaceutical Industry, Barry Wintner, Anthony Contino, Gary O'Neill. BioProcess International DECEMBER 2005].

BASF has shown that chlorine dioxide is also effective against human coronavirus [Aseptrol S10-Tab (70060-19)—Notification Sep. 2, 2015].

An antiviral mechanism of action of ClO2, which is known in the art, concerns the guanine bases, which occur as a component of the nucleotides in the RNA and DNA chains of viruses. Guanine is very sensitive to oxidation. If the ClO2 molecule comes into contact with and oxidizes guanine, this leads to the formation of 8-oxoguanine, which affects the multiplication of the nucleic acids already at the transcription stage and subsequently prevents the formation of a fully functional virus.

It is the object of the present invention, in view of the current emergency situation with COVID-19, to provide an inexpensive therapeutic aid in the form of a chlorine dioxide solution for use in the therapeutic treatment of COVID-19 patients, in particular in the form of an aqueous solution of chlorine dioxide for oral and/or intravenous administration to infected patients.

This object is achieved with a solution according to the independent claim. Further advantageous embodiments of the invention are described in the dependent claims.

There are now numerous studies available on the possible toxicity of chlorine dioxide, all of which show that chlorine dioxide can be safely used in therapeutically suitable doses, see for example [2017, Ma et al., Efficacy and Safety Evaluation of a Chlorine Dioxide Solution]. The lethal dose LD50 for humans is considered to be the intake of 292 mg per kilogram of body weight per day over a period of 14 days.

The dosage recommended according to the present invention for oral administration, on the other hand, is only 1-10 mg of ClO2 per kg of body weight per day, that is only a maximum of 1/30 of the LD50, and is preferably administered in the form of 5-10 dose units of 100 ml of ClO2 solution a 10-75 mg, in particular a 20-50 mg, of dissolved ClO2.

In one embodiment, the invention relates to a pharmaceutical composition which contains high-purity chlorine dioxide (ClO2) in an aqueous medium and is suitable for systemic, parenteral use for the treatment of COVID-19 patients.

This composition contains dissolved ClO2 in a concentration of typically 5 to 1000 ppm, corresponding to 5 to 1000 mg/I. However, a concentration range of 10-500, especially 25-300 ppm is most suitable for most applications.

In this context, “high purity” means that the composition is substantially free from chlorine gas, hydrochloric acid, chlorite, and chlorate ions, i.e. these components typically each contain only concentrations of less than 1% of the ClO2 concentration. This is achieved, for example, by means of a production method as described in WO 2018/185346. The concentrations of Na chlorate, Na chlorite, chlorine gas, and hydrochloric acid are in the freshly produced, concentrated CDL, which usually contains 1000-2000 ppm (mg/L) ClO2, typically in a range of at most 10-20 ppm (mg/L) and in the ready-to-use, diluted CDL at a maximum of 1-2 ppm (mg/L) or below. If necessary, the ClO2 solution can also be produced with higher levels of 3000 ppm and more of ClO2.

The main advantage of a high-purity CDL is that the likelihood of susceptible undesirable effects, which are described for Na chlorite and especially for Na chlorate in the specialist literature, is reduced as much as possible, which is particularly important for formulations and compositions that should be used as infusion solutions or injection solutions.

A composition according to the invention, which, in addition to external, topical application, is intended to be particularly suitable for systemic, in particular parenteral, applications in the form of injection or infusion solutions, can contain at least one tonicity regulator which makes it possible to adjust the solution isotonic. Tonicity regulators that can be used are ionic substances such as NaCl or KCl, but also non-ionic substances and here, above all, representatives from the group of monosaccharides, disaccharides, oligosaccharides, and low molecular weight polyols. The mono- and disaccharides are preferably selected from the group of: glucose, fructose, sucrose and mannose and the low molecular weight polyols are preferably selected from the group of: glycerol, erythritol, lactitol, mannitol, sorbitol, inositol, xylitol, threitol and maltitol.

An isotonic saline solution contains 9 g NaCl per liter of water (0.9%), the resulting osmolarity of 290-300 mosmol/l substantially corresponds to the osmolarity of the blood, which can also be adjusted with KCl or a mixture of two or more of the ionic and non-ionic tonicity regulators.

Especially for injection and infusion solutions, it can be of crucial importance to adjust the pH of the CDL to that of the blood in a range of approx. pH 7.3-7.5 in order to avoid any undesirable effects. For this purpose, a pH regulator, in particular a buffer system, can be added to the CDL. A bicarbonate buffer or a phosphate buffer, for example PBS, is considered suitable. Even though studies in vitro have shown that a higher or lower pH of CDL would be advantageous for some applications, compatibility with blood pH should be given priority for injection and infusion solutions. For other applications, for example oral applications, higher or possibly lower pH values can also be set.

The addition of further components from the group of preservatives, flavors, taste-masking substances, vitamins, mineral salts, and trace elements can be advantageous for the compositions according to the invention, in particular those which are intended for prolonged, recurring use.

For external use for surface disinfection of, for example, hands, equipment, protective clothing, furniture, and the like, the ClO2 solution does not necessarily have to meet the criteria mentioned above for a high-purity solution. A commercially available quality may also be sufficient for these purposes. The same applies to solutions for oral administration, although a high-purity ClO2 solution is preferred here.

In line with the reaction kinetic knowledge from the topical application of ClO2 solutions by Noszticzius et al., it was possible to confirm in the course of the present invention that, contrary to the earlier fears of parts of the professional world, a systemic therapeutic use of reactive oxidative species such as ClO2 need not in any way be accompanied by undesirable or even damaging effects. In fact, no such reports of serious side effects or even deaths seem to have been found in the literature to date, and no such effects have come to light in the course of use in the context of the present invention.

The following examples are intended to illustrate the invention further and to underpin the therapeutic applicability of the oxidative intervention by means of chlorine dioxide solution. At this point, it should also be pointed out that the treatments described in the following patient examples were carried out either by doctors or by trained personnel under medical supervision.

### Example 1: Increase in Oxygen Partial Pressure in the Blood

Three test subjects aged 32-57 years were each given 250-500 ml of the high-purity ClO2 solution with a concentration of 50 ppm intravenously over a period of 3 hours. A blood count was taken 30-120 minutes after the end of the infusion and, among other things, the blood gases of a venous blood sample were measured.

| | pO2 before | ClO2 | Time after | pO2 after |
|---|---|---|---|---|
| | ClO2 infusion | solution | infusion | ClO2 infusion |
| | [mmHg] | 50 ppm [ml] | [min] | [mmHg] |
| Patient 32 | 34.0 | 500 | 122 | 57.8 |
| years | | | | |
| Patient 54 | 35.6 | 500 | 67 | 40.0 |
| years | | | | |
| Patient 57 | 23.2 | 250 | 25 | 30.0 |
| years | | | | |

Conclusion:

The intravenous administration of chlorine dioxide solution caused an increase in the oxygen partial pressure in the venous blood in all three test subjects. Chlorine dioxide doses seem to have a positive influence on the oxygen supply to the lungs and/or cell respiration in the mitochondria, i.e. to improve them. An effect that can be of decisive advantage, particularly in the case of viral infection of the upper airways and lungs, and enables affected patients to recover more quickly.

### Example 2: Treatment Protocols with ClO2 Solution (CDL) in SARS-CoV-2 Infections

The following treatment protocols are proposed from a medical point of view and based on the long-term experience of the inventor in the therapeutic use of low-dose chlorine dioxide solution for the treatment of coronavirus infections, in particular SARS-CoV-2 infections.

Precautions and Contraindications

1. As an oxidizing agent, the effectiveness of chlorine dioxide in eliminating pathogens is eliminated by taking vitamin C and other antioxidants at the same time;

2. Allow 1 hour to take medication and 1/2 hour to take meals.

3. The concentrated CDL must be stored refrigerated below 11° C. Protect from UV light.

4. CDL is an oxidizing agent that easily attacks metals. This must be taken into account when storing and washing devices and materials.

5. If the effect is too aggressive on contact in the area of mucous membranes with a concentrated formulation, then it should be diluted to 50 mg ClO2/I (0.005%) with physiological solution.

6. CDL in a concentrated form discolors the tissue that oxidizes it

7. ClO2 should NOT be inhaled in concentrated doses (lung toxicity)

8. In patients treated with warfarin, the values should be constantly checked to avoid cases of overdose. Chlorine dioxide has been shown to improve blood circulation.

Protocol D

Dermatological Applications, Hand and Surface Disinfection, with >1000 ppm ClO2 Solution

This protocol is used to disinfect both the skin and objects that could be infected. It consists of using a sprayer filled with CDL concentrate from 1000 to 2000 ppm (i.e. 0.1 to 0.2% ClO2).
Apply the spray directly to the desired area and rub it in gently; it is used like a hydroalcoholic gel.

For sensitive areas such as eyes and mucous membranes, it is necessary to reduce the ClO2 concentration to about 50 ppm with water or physiological saline, which is still sufficient to deactivate pathogens.

Protocol H

Used to disinfect rooms, for example in hospital rooms, to avoid infection between patients and medical personnel

10 ml of concentrated 0.3% CDL are placed in a dry beaker and placed between the patient beds. ClO2 gas (boiling point 11 degrees C.) evaporates due to the temperature of the room and disinfects the surroundings, thereby avoiding infection of the patient and the health personnel in the same room.

A saturated chlorine dioxide solution has a yellowish color, which subsequently fades as the gas evaporates until the liquid in the glass becomes colorless.

The released ClO2 gas is distributed evenly throughout the room.

According to calculations, a room of around 12 square meters can already be successfully disinfected with a CDL solution of 1 ppm, which complies with the safety regulations and international toxicology regulations and is approved for use

Protocol C

Usually used for prevention in health care (protection of medical personnel) and for the treatment of asymptomatic, SARS-CoV-2 positive patients; 10 dose units are provided

This protocol serves as a preventive measure, both for personal use in health care and for the treatment of positive asymptomatic patients.

1. Dilute 10 ml of concentrated 3000 ppm CDL in 1 liter of water.

2. Divide the diluted solution into 10 dosing units of 100 ml each and drink about one dosing unit per hour until the 1000 ml are used up.

Acute Treatment

The same protocol can be used in the event of a severe course of the disease or even a risk to life, but the dose should be increased slowly if necessary until 30 ml of concentrated CDL per liter of water are reached.

Protocol F

This protocol is proposed to combat viral infections and acute bacterial infections:

Dosage: 8-12×1 ml conc. Take CDL at 15-30 minute intervals=8 to 12 ml conc. CDL (3000 ppm), whereby 1 ml of concentrated CDL (0.3%) should be diluted to 100 ml with drinking water.

Alternatively, 8-12 ml of concentrated CDL (0.3%) can be added to a bottle and made up to 1 L with drinking water. Divide the contents of the bottle into 10 equal parts of 100 ml or mark the bottle with appropriate markings, and take a 100 ml dosage unit every fifteen to thirty minutes.

Depending on the severity of the disease, protocol F can be carried out once or twice a day:
if 2×/day: preferably in the morning and in the afternoon, with a time interval of at least 2 hours between the two intake cycles.
if 1×/day: adjust the dosage according to protocol C if necessary.

Protocol Y

This protocol is used for injections and infusions
Protocol C should generally have been run at least once before starting parenteral therapy.
Creation of a blood picture including measurement of the venous blood gas values in order to determine the current condition of the patient
Preparation: Add 1-2 ml concentrated CDL (0.3%) per 100 ml isotonic physiological saline. A typical adult dose is 5 ml CDL (0.3%) in 500 ml 0.9% NaCl solution (30 ppm CLD), corresponding to 15 mg ClO2 total dose; if necessary, increase the dose to double.
Measure the pH value of the CLD, which must be between pH 7.4 and pH 7.8; if it is below, buffer with sodium bicarbonate;
Slowly set the drip speed for iv infusion; best between 2 and 4 hours of dripping with 500 ml solution for infusion;
perform blood gas determination again after CDL infusion;
typical duration of treatment for current COVID-19 symptoms: 4 consecutive days; a different route on a different extremity should be chosen every day;
after the 4-day infusion treatment, if necessary, can continue with protocol C until the patient is symptom-free.

### Example 3: Patient with COVID-19

A 26-year-old SARS-CoV-2 positive patient was treated with ClO2 solution (500 ml, 50 ppm; oral administration in 5 units of 100 ml at 1 hour intervals). After 3 days, no virus was detectable in the respiratory secretion, taken from the pharynx, using the RT-PCR method.

### Example 4: Male Patient Infected with SARS-CoV-2

On 4 consecutive days, the patient received 1 treatment cycle of 10 dosage units, each containing 100 ml aqueous ClO2 solution 30 ppm (30 mg ClO2/liter). The dosing units were administered orally at 1 hour intervals.

Analysis Method:

RNA extraction: RNA extraction was performed from 500 ul sample (exudate).

Molecular Detection:

A real-time qualitative PCR test was used to detect the SARS-CoV-2 virus (Covid-19). Briefly, RNA is extracted from the sample, transcribed back into cDNA and amplified using RealTime (RT)-PCR and detected with specific probes.

Result:

After 4 days of therapy with chlorine dioxide solution, the presence of SARS-CoV-2-RNA could no longer be demonstrated. All in all, the result was NEGATIVE for Covid-19.

### Example 5: Cohort Study to Determine the Effectiveness of Oral Chlorine Dioxide in the Treatment of COVID-19

The aim of this study is to use prospective case research to test the effectiveness of oral chlorine dioxide in the treatment of patients with COVID infection 19. The research will be conducted between April and June 2020 with a quasi-experimental design in two therapy centers on a sample of twenty (20) patients through direct intervention that changes the symptoms of the infection and the absence of COVID-19. Measure infection after administration of the study preparation chlorine dioxide to determine the effectiveness of chlorine dioxide in the treated group.

Based on the results found and the assessment of efficacy based on the clinical improvement on a scale of 1 to 5 and the absence (“negation”) of COVID-19, it can be deduced whether the therapeutic efficacy considered in this study can be viewed as good, i.e. whether the treatment with chlorine dioxide in COVID-19 is effective or not.

This research aims to stimulate the search for new therapeutic options in the treatment of COVID-19 and to contribute to the development of new avenues in drug therapy in general, particularly given the large number of deaths and the relatively high mortality rate of the current SARS-CoV-2 pandemic.

Cohort/Patient Group

Study drug: The starting material is a concentrated aqueous chlorine dioxide solution with a dissolved ClO2 content of 3000 mg/L (3000 ppm); 1 bottle contains 150 ml.

Allocation of Study Medication

Each patient receives a consecutive patient number and the corresponding study medication in the order in which they were admitted to the study. The assignment of this drug was made before the start of the study using a computer-generated list.

Dosage and Route of Administration

Drug: The initial preparation is an aqueous chlorine dioxide solution 3000 ppm, 1 bottle of 150 ml. 10 ml of chlorine dioxide solution (3000 ppm) are diluted with 1 liter of water to approx. 30 ppm per day. Each patient receives the starting drug along with genquen, written instructions for making and taking the dilutions. A dosage unit of approx. 80-120 ml of the diluted chlorine dioxide solution (approx. 30 ppm) is taken orally every hour until the contents of the bottle are used up (8 to 12 doses).

Both the concentrated starting solution (3000 ppm) and the diluted experimental chlorine dioxide solutions (approx. 30 ppm) should be kept refrigerated.

Study Participants:

The study participants were a group of medical and healthcare patients with active COVID-19 infection from various medical care centers and hospitals in the cities of Bogota, Colombia and Madrid, Spain (multicentre). The selection of patients was made on the basis of the doctors' and patients' own suggestions to be allowed to participate as candidates for research. Simultaneity was also applied, which means that all patients were selected from the same time interval of the spreading pandemic.

Criteria

Inclusion Criteria:

a) Individuals who tested positive for Covid-19

b) Individuals who have some typical symptoms of Covid-19 such as fever, difficulty swallowing (odynophagia), shortness of breath.

c) Individuals aged 18 to 80

Exclusion Criteria:

a) Individuals who tested negative for Covid-19

b) Individuals with stage IV/VI renal insufficiency

c) Individuals with heart failure

d) Patients taking anticoagulants (“blood thinners”), especially those based on warfarin sodium

Primary Assessment of the Results Based on:
negative test of Covid-19 over a period of 7 days
Amplification of coronavirus genes by real-time (RT)-PCR

Interim Results:

After 4 days of treatment with chlorine dioxide solution, most of the typical symptoms of the disease in the group of infected persons had disappeared and COVID-19 could no longer be diagnosed.

### Example 6 Treatment with CDL on 104 Subjects

The test group included individuals who tested positive for COVID-19 as well as their family members and individuals who had been in contact with COVID-19 positive patients for a long time and who then got the symptoms.

The symptoms and their changes in the course of treatment with oral CDL therapy are shown in Tables 1 to 4 below. The test group was treated by 5 doctors, who had successfully treated their own infection with the SARS-CoV-2 virus with chlorine dioxide solution beforehand and achieved a practically complete reduction in the typical symptoms of the disease. Ultimately, this success rate was the reason for further clinical examinations with this preparation under conditions where every single patient is tested very carefully. The investigation took place in the city of Guayaquil in Ecuador, a hotspot of the pandemic in Latin America.

Treatment Schedule:

10-20 ml of an aqueous 3000 ppm chlorine dioxide solution were made up to 1 liter in a bottle with water, which corresponds to a ClO2 content of the dilute chlorine dioxide solution of 30-60 ppm of dissolved ClO2. The patients drank this water throughout the day in ten units of approx. 100 ml and had extreme improvements after a very short time. After four days, the majority of the 100 participants in this test were practically symptom-free, including the typical Covid-19 symptoms of the lungs and hypoxia.

TABLE 1
Day 1

| Symptoms | Women | Men | TOTAL |
|---|---|---|---|
| Fever | 40 | 28 | 68 |
| Chills | 16 | 14 | 30 |
| Muscle aches | 41 | 31 | 72 |
| Dry cough | 45 | 38 | 83 |
| Headache | 37 | 38 | 75 |
| Back pain | 40 | 38 | 78 |
| Difficulty breathing | 17 | 22 | 39 |
| Vomit | 3 | 10 | 13 |
| Diarrhea | 12 | 17 | 29 |
| Sore throat | 41 | 40 | 81 |
| Loss of smell | 49 | 40 | 89 |
| Loss of taste | 36 | 33 | 69 |
| Lack of appetite | 32 | 25 | 57 |

TABLE 2
Day 2

| Symptoms | Women | Men | TOTAL |
|---|---|---|---|
| Fever | 12 | 11 | 23 |
| Chills | 3 | 6 | 9 |
| Muscle aches | 35 | 31 | 66 |
| Dry cough | 41 | 37 | 78 |
| Headache | 14 | 15 | 29 |
| Back pain | 37 | 31 | 68 |
| Difficulty breathing | 12 | 18 | 30 |
| Vomit | 2 | 5 | 7 |
| Diarrhea | 4 | 10 | 14 |
| Sore throat | 14 | 24 | 38 |
| Loss of smell | 44 | 38 | 82 |
| Loss of taste | 18 | 22 | 40 |
| Lack of appetite | 13 | 14 | 27 |

After the second day of treatment, massive improvements in health status can be seen. In particular, fever, chills, headache, sore throat and loss of appetite have disappeared in most patients.

TABLE 3
Day 3

| Symptoms | Women | Men | TOTAL |
|---|---|---|---|
| Fever | 2 | 3 | 5 |
| Chills | 1 | 2 | 3 |
| Muscle aches | 23 | 25 | 48 |
| Dry cough | 39 | 37 | 76 |
| Headache | 3 | 4 | 7 |
| Back pain | 33 | 29 | 62 |
| Difficulty breathing | 5 | 10 | 15 |
| Vomit | 1 | 2 | 3 |
| Diarrhea | 3 | 5 | 8 |
| Sore throat | 5 | 10 | 15 |
| Loss of smell | 13 | 19 | 32 |
| Loss of taste | 3 | 5 | 8 |
| Lack of appetite | 4 | 4 | 8 |

TABLE 4
Day 4

| Symptoms | Women | Men | TOTAL |
|---|---|---|---|
| Fever | 1 | 0 | 1 |
| Chills | 0 | 1 | 1 |
| Muscle aches | 14 | 16 | 30 |
| Dry cough | 35 | 27 | 62 |
| Headache | 1 | 1 | 2 |
| Back pain | 22 | 27 | 49 |
| Difficulty breathing | 2 | 4 | 6 |
| Vomit | 0 | 0 | 0 |
| Diarrhea | 3 | 1 | 4 |
| Sore throat | 0 | 1 | 1 |
| Loss of smell | 5 | 3 | 8 |
| Loss of taste | 1 | 0 | 1 |
| Lack of appetite | 2 | 0 | 2 |

After 4 days of CDL treatment, apart from muscle pain, dry cough and back pain, all other symptoms have disappeared in almost all patients.

TABLE 5
Distribution of patients by age group

| Distribution of participants by age group | | | | | | |
|---|---|---|---|---|---|---|
| | Gender | | | | | |
| Age | Female | % | Male | % | TOTAL | Percent |
| >10 years | 3 | 2.9 | 4 | 3.8 | 7 | 6.7 |
| 11-20 | 6 | 5.8 | 3 | 2.9 | 9 | 8.7 |
| 21-30 | 7 | 6.7 | 7 | 6.7 | 14 | 13.5 |
| 31-40 | 12 | 11.5 | 7 | 6.7 | 19 | 18.3 |
| 41-50 | 9 | 8.7 | 13 | 12.5 | 22 | 21.2 |
| 51-60 | 7 | 6.7 | 10 | 9.6 | 17 | 16.3 |
| 61-70 | 5 | 4.8 | 3 | 2.9 | 8 | 7.7 |
| 71-80 | 5 | 4.8 | 2 | 1.9 | 7 | 6.7 |
| <80 | 1 | 1.0 | 0 | 0.0 | 1 | 1.0 |
| TOTAL | 55 | 52.9 | 49 | 47.1 | 104 | 100.0 |

The distribution of patients by age and gender almost corresponds to an ideal Gaussian distribution.

At this point it should be pointed out that in addition to the effects of chlorine dioxide administration mentioned at the outset, other, less well-studied effects may also play a role. In particular, an increasing number of findings indicate that the administration of the ClO2 solution leads to an increase in the oxygen partial pressure in the blood capillaries and generally in tissues poorly supplied with blood, and in particular that oxygen that is urgently needed for the mitochondria can thus be made available immediately. This could be of enormous therapeutic benefit, especially for immunocompromised patients and generally for physically weakened and bedridden patients who suffer from COVID-19 and may even make the recovery of these patients possible in the first place. Plants have already shown that chlorine dioxide has a marked influence on the redox status, the NAD/NADH ratio, and ATP production in mitochondria (see e.g. Chumyam et al., Effects of chlorine dioxide on mitochondrial energy levels and redox status of “Daw” longan pericarp during storage; Postharvest Biology and Technology, Volume 116, June 2016, pages 26-35).

1. Chlorine dioxide in aqueous solution, for use as an antiviral agent in the preventive and/or therapeutic treatment of viral infections of the human or animal body caused by SARS-CoV-2 coronaviruses.

2. Chlorine dioxide solution according to claim 1, characterized in that it contains 5 to 1000 mg/l (ppm) of dissolved chlorine dioxide (ClO2).

3. Chlorine dioxide solution according to claim 1 or 2, characterized in that it is free from chlorate ions, hydrochloric acid, and gaseous chlorine in the ready-to-use state or contains these components in a concentration of at most 1% of the chlorine dioxide concentration.

4. Chlorine dioxide solution according to any of claims 1 to 3, characterized in that it is sterile and pyrogen-free and contains one or more of the following components:
a) 3 to 10 g/l of an ionic tonicity regulator from the group NaCl and KCl; or a non-ionic tonicity regulator from the group consisting of monosaccharides, disaccharides, oligosaccharides, and low molecular weight polyols; or a mixture of the aforementioned components;
b) a pH regulator in the form of a pH buffer system, set to a value in the range from pH 5.0 to 7.5;
c) 0.1 to 20 g/l dimethyl sulfoxide (DMSO) or dimethyl sulfone (MSM).

5. Chlorine dioxide solution according to claim 4, characterized in that as a non-ionic tonicity regulator at least one mono- or disaccharide is contained from the group of: glucose, fructose, sucrose, mannose, and/or at least one low molecular weight polylol is contained from the group of: glycerol, erythritol, lactitol, mannitol, sorbitol, inositol, xylitol, threitol and maltitol.

6. Chlorine dioxide solution according to any of claim 1 or 2, for use in the disinfection of surfaces including human skin.

7. Chlorine dioxide solution according to any of claim 1 or 2, for use in the disinfection of room air, in particular in patient rooms and treatment rooms in hospitals and similar sanitary facilities.

8. Chlorine dioxide solution according to any of claims 1 to 5, adapted for oral use.

9. Chlorine dioxide solution according to any of claims 3 to 5, adapted as an isotonic solution, for use as a solution for injection or infusion.

10. Pharmaceutical composition based on the chlorine dioxide solution according to any of claims 1 to 5, for use as a therapeutic agent in the oral and/or parenteral, systemic treatment of viral infections of the human or animal body, caused by coronaviruses of the type SARS-CoV-2.
